(19) United States
(12) Patent Application Publication
(10) Pub. No.: US 2004/0048380 A1
(43) Pub. Date: Mar. 11, 2004

(54) Chimeric vectors and their use for heterologous genes transfer

(76) Inventors: Isabella Saggio, Ventotene (IT); Barbara Salone, Montesilvano (IT); Monica Di Giovine, Rome (IT); Martina Yuri, Aprilia (IT)

Correspondence Address: WILLIAM COLLARD, COLLARD & ROE, P.C., 1077 NORTHERN BOULEVARD, ROSLYN, NY, 11576, US

(21) Appl. No.: 10/381,182

(22) PCT Filed: Sep. 21, 2001
(86) PCT No.: PCT/IB01/01742

(51) Int. Cl. C12N 15/86; C12N 15/74; C12N 7/00

(52) U.S. Cl. 435/456; 435/235.1; 435/472

(57) ABSTRACT

The present invention relates to chimeric biological vectors for gene trnasduction in eukaryotic cells. The invention further relates to a method for producing said chimerized vectors and a mehod for transduction of eukaryotic cells, in particular cells expressing integrin receptors.

### FIELD OF THE INVENTION

The technical field of the present invention is the engineering of biological vectors for applications in the field of gene therapy.

### PRIOR ART

The possibility to transfer exogenous DNA into cells, in particular into eukaryotic cells, effectively and safely is one of the main aims of bio-technological research. At present, researches carried out in this area are focusing on various features, namely by making traditional chemical (DEAE-dextrane, calcium-phosphate) or mechanical (e.g. microinjection, electroporation) systems more efficient, by developing new systems such artificial lipids, dendrimers or in general artificial polymers, and by engineering new biological vectors.

As summarized in Luo et al. (Luo D., Saltzman W. Nature Biotech 2000; 18: 33-37) the transduction is globally efficient if the single steps constituting it are efficient, and in particular if: a) the bond to the target cell is efficient, b) the cytoplasmic transport and the protection of DNA from cytoplasmic endonucleases are efficient, c) if DNA is efficiently vehiculated to the nucleus, and eventually, d) if the latter is efficiently transcribed. Gene transfer mediated by biological vectors, mainly viruses, retroviruses, adenoviruses and their derivates, enables the intra-cellular and then the intra-nuclear delivery of the therapeutic gene much more efficiently than other DNA vehiculating systems.

Vectors based on eukaryotic virus systems require quite a hard and complex preparation and purification, since their amplification for the uses related with gene therapy has to be carried out in eukaryotic cells in culture. The engineering of these viruses is also highly complex; indeed, changes made by introducing exogenous sequences can often decrease their infectivity and the effectiveness of the gene transfer vector. The use of eukaryotic viruses is further strongly limited by the difficulty in orienting said viral vectors to new specific cell targets while eliminating the natural tropism of the virus. As a matter of fact, the elimination of the latter is complicated by the presence of redundant ways of cell recognition, developed by the virus during its evolution.

Other problems related to the use of viral vectors are: the possibility of recombination of the viral genome with the cellular genome and the often incomplete inactivation of viral genes which can be potentially dangerous for the cells, as observed for adenoviral vectors by Yang et al. (Proc. Natl. Acad. Sci. USA, 91, 4407-4411).

However, eukaryotic viruses represent, under various points of view, a model to take inspiration from for optimizing second-generation and third-generation “synthetic” or biological systems.

Recent studies have shown that it is possible to modify the natural targeting specificity of bacteriophages and in particular of filamentous phages, which in nature bind and infect only prokaryotic cells, by the functional display of ligands of eukaryotic receptors on the capsid structure of the phage. The same authors of the present application have shown that, when the sequence of CNTF (Ciliary Neurotrophic Factor) is expressed on the phage capsid, the phage particle gain the ability to bind specifically eukaryotic cells expressing CTNF-R (Saggio I. et al. Embo. J. 1995; 14: 3045-3054). Studies carried out by other groups of researchers have also shown that the functional display of ligands for cell receptors such as FGF-R (Larocca D. et al. FASEB J. 1999; 6: 727-734 and WO99/10014) and erbB2, receptor of EGF (Poul M., Marks J., J. Mol. Biol. 1999; 288: 203-211 and WO 99/55720) not only determine the binding specificity of the phage but also enable the internalization of the phage particle and the weak expression of a reporter gene.

Until now the optimization of these “artificial” biological systems has been mainly aimed at optimizing the targeting stage, i.e. the first step of the transduction mechanism. Only recently, and as a consequence of the studies carried out on purified viral proteins, such as for instance on the VP22 of herpes virus (Elliott G. Gene Ther. 1999, 6:149-151) or on the proteins of the adenoviral pentameric complex (WO 94/17832), it is possible to clarify also the mechanisms activated in the steps following the binding to the eukaryotic cell, said mechanisms being used by viruses in the naturally efficient infection process, with great advantages concerning the development of artificial gene-vehiculating systems.

In the field of gene therapy, however, it is still particularly necessary to provide for effective gene transfer systems with the binding specificity of eukaryotic viruses, though being easily engineered and produced as bacteriophages.

### SUMMARY

The main object of the present patent application is represented by bacteriophage vectors for the gene transduction of eukaryotic cells, characterized in that they have on their outer surface, beyond structural proteins typical of phages, viral proteins or polypeptides of eukaryotic viruses. The bacteriophage vectors also comprise a therapeutic gene under the control of a transcription promoter and are obtained by genetic engineering of bacteriophage vectors, such as λ or M13, or by cross-linking of phage particles with chemical agents.

The viral proteins or polypeptides which are used are: HA protein (hemoagglutinin) of influenza virus, protein VP22 of Herpes simplex virus, penton-base adenoviral protein or its central loop, comprising amino acids 295-380, or fragments of such proteins/polypeptides.

The authors of the present application have now surprisingly found that chimeric bacteriophage vectors not only acquire the binding specificity of the viral protein used for chimerization, by specifically binding to the eukaryotic cell, but they can also activate the internalization way which is typical of the virus as a whole, thus optimizing the vehiculation of the therapeutic gene, of the reporter gene or of heterologous nucleic acids, through the cytoplasm to the nucleus.

According to another aspect, the invention relates to a process for producing bacteriophage vectors chimerized with viral polypeptides or proteins derived from eukaryotic viruses and to the products obtained from said process.

According to another embodiment, the invention relates to a process for the transduction of eukaryotic cells, in particular of cells expressing integrin receptors.

A further embodiment of the invention also provides for a process for identifying binding and internalization mutants of the penton-base adenoviral protein by using chimeric bacteriophage vectors.

Another embodiment of the invention relates to compositions comprising a physiologically acceptable liquid and a bacteriophage vector for gene transfer into eukaryotes, chimerized with viral proteins or polypeptides among which: HA protein (hemoagglutinin) of influenza virus, VP22 protein of Herpes simplex virus, penton-base adenoviral protein, or their fragments or mutants.

A final feature of the invention relates to the use of the nucleotide sequence encoding penton-base adenoviral protein or its fragments, and of the sequence comprising amino acids 295-380 of penton-base adenoviral protein for engineering chimeric transduction vectors for eukaryotic cells.

### DESCRIPTION OF THE FIGURES

FIG. 1. In-vitro binding of chimeric filamentous phages to integrins. Integrins were immobilized on a plate and incubated with Pb phage (4×10¹² particles/well), with ΔPb phage (1×10¹² particles/well), or with control phage (4×10¹² particles/well). The bound phages were detected with a primary anti-M13 antibody and with a secondary anti-pVIII-HRP antibody. Data are shown as average OD values from tests carried out in two series. Standard deviation (SD) is also shown.

FIG. 2. Electron microscopy for the detection of the binding of chimeric phages to HeLa cells. 10⁵ HeLa cells are incubated at 4° C. with 3×10¹² and 9×10¹² particles of ΔPb phage (B, C) and Pb phage (A), respectively. After incubation the cells are treated as for analysis by electron microscope. Original enlargement: A, 15500×; B, 5200×; C, 11500×.

FIG. 3. Internalization of chimeric phages into eukaryotic cells, detected by immunofluorescence. 2.5×10⁵ HeLa cells are incubated with chimeric phage particles. Panel C, control phage (3×10¹² particles/well); panels B, D and E: ΔPb phage (3×10¹² particles/well); panel A: Pb phage (9×10¹² particles/well). Panels A, B e C: incubation for 1 hour at 4° C., followed by 1 hour at 37° C.; panels D and E: incubation for only 1 hour at 4° C. to inhibit internalization due to the receptor. The cells are observed with a fluorescence microscope with a 40× objective.

FIG. 4. Effect of the inhibitors of Wortammanin and ML-7 kinases on cell internalization. 10¹² particles of ΔPb phage (white bars) or of Pb phage (striped bars) are adsorbed on HeLa cells. The internalized phage is recovered and titrated. The figure also shows the tests carried out in presence of inhibitors of the kinases Wortammanin (WTN, 1 μM) and ML-7 chloride (ML-7, 2 μM). Data are shown as percentages of control enrichments carried out without inhibitors. Th results are average values of three different tests carried out in two series; SD (Standard Deviation) is also shown.

FIG. 5. Chimeric phages transduce eukaryotic cells in a receptor-dependent way. Cells are incubated for 1 hour at 4° C. and for 3 hours at 37° C. with 2×10¹³ particles of Pb-GFP phage or ΔPb-GFP phage. After 72 hours said cells are analyzed with FACS. For competition tests they are pre-incubated with GRGDSP or GRGESP peptides (4.86 μM, corresponding to a molar excess of about 2000 times) for 1 hour at 4° C. and then incubated with chimeric phages. Data are analyzed with WinMDI2.8 software. 10⁴ cells for each well are counted. a) White bars correspond to ΔPb-GFP phages; b) striped bars correspond to Pb-GFP b) phage.

FIG. 6. In-vitro binding of chimeric lambda phages to integrins. Plates, where ανβ3 integrin receptor has been immobilized, are incubated with M13ΔPb phage (1×10¹² particles/well), or with λΔPb phage (1×10⁸ and 1×10⁹ particles/well), detected with a primary anti-M13 antibody and with a primary anti-λ-phage rabbit polyclonal antibody, respectively, then with a secondary anti-pVIII-HRP antibody and with an anti-rabbit HRP antibody (Amersham-Pharmacia Biotech), respectively. Data are shown as average OD values from tests carried out on two series.

### DETAILED DESCRIPTION OF THE INVENTION

The main object of the present patent application is represented by bacteriophage vectors for gene transduction of eukaryotic cells, characterized in that they have on their outer surface, beyond structural proteins typical of phages, viral proteins or polypeptides of eukaryotic viruses. Said vectors are therefore defined as chimeric. They can be obtained by chemical methods, for instance by conjugating viral proteins with the outer structure of the bacteriophage through cross-linking agents, or preferably by genetic engineering techniques using basic bacteriophage vectors consisting of: bacteriophages, phagemids or plasmids, such as those used in the prior art for cloning, sub-cloning or mutagenesis purposes, such as for instance lambda phage, or filamentous bacteriophages belonging to M13 family. Since the genome of the bacteriophage is found in a plasmidic or a phagemidic form, the expression “chimeric bacteriophage” refers to any embodiment in which the DNA sequence encoding a phagic protein is chimerized with the DNA sequence encoding for a surface structural protein derived from an eukaryotic virus, said proteins being contained in a plasmid, bacteriophage or phagemid vectors, or, alternatively a bacteriophage particle displaying on its surface a structural protein or a protein of viral origin (from an eukaryotic virus) either obtained by chimerization of phage proteins or by assembly of proteins differently expressed within the bacterial host cell.

Similarly to eukaryotic viruses, bacteriophages consist of an outer structure, mainly a proteic structure, and of inner proteins, having for instance packaging function for the single-helix or double-helix nucleic acids which constitute the genome of the bacteriophage. In the framework of the present invention, the expression “bacteriophage vector” refers both to the “nude” DNA (in linear or circular form) encoding secreted, extruded or differently obtained—for instance by lysis—phage particles, and to the bacteriophage particle, which consists both of the polynucleotidic component of the genome, the latter being recombinant or not, and of the inner and outer proteic component.

The basic bacteriophage vectors or phagemids used in the present invention consist in their turn of the genome of the bacteriophage, integral or partially deleted, plus synthetic or natural accessory sequences, such as for instance cloning sites, resistances to antibiotics etc., which are added in order to be used in laboratory as cloning, sub-cloning and mutagenesis vectors (Sambrook, J., E. F. Fritsch and T. Maniatis. 1989. Molecular cloning: a laboratory manual, 2^{nd} edition, Cold Spring Harbor Laboratory Press, N.Y.).

According to a preferred embodiment of the invention the chimeric bacteriophage vectors according to the present invention, are obtained by genetic modifications of the basic bacteriophage vector, which comprise the partial or complete replacement of the natural sequence encoding at least one of the surface structural proteins of the bacteriophage with sequences encoding viral proteins or polypeptides. In the case of chimeric vectors obtained by genetic engineering techniques, both the outer proteic structure of the bacteriophage and its genom are chimerized with exogenous viral nucleotide or amino acid sequences. Th methods to genetically manipulate bacteriophages in order to display peptides or heterologous proteins on the outer surface are known in the state of the art (Hoogenbom H. R. et al. Nucleic Acid Res. 1991, 19:4133-4137 e U.S. Pat. No. 5,736,388).

The chimerized bacteriophage vectors according to the invention, show several advantages with respect to vectors for the transduction of eukaryotic cells known in the state of the art: they can be amplified and produced in their natural hosts, i.e. bacteria, thus greatly simplifying all the steps which are necessary in order to obtain high-titer virus stocks and which are carried out on mammalian cells for eukaryotic viral vectors (retrovirus vectors, adenovirus vectors, etc.). They are safer because, since their natural hosts are bacteria, they do not have sequences which might potentially interfere with eukaryotic cell functions, and which might therefore be potentially dangerous, as it happens for viruses whose natural hosts are humans and mammals.

The chimerized bacteriophage vectors according to the invention further comprise a therapeutic gene under the control of a transcription promoter. A therapeutic gene is any sequence of heterologous DNA whose translation, transcription or introduction into the genome of the host cell results in a therapeutic effect within said cell, in the surrounding tissue or in the host organism. In addition, heterologous DNA sequences encoding for proteins used as cell markers, such as for instance the sequences encoding for the Green Fluorescent Protein (GFP), β-galactosidase or luciferase, or sequences encoding the resistance to selective agents such as HPRT, geneticin TK, MDR, hygromycin may be used.

Said therapeutic genes, or sequences of heterologous DNA, are preferably introduced into an expression box, i.e. under the control of regulatory sequences: such as for instance transcription enhancers and promoters either constitutive or inducible, viral or cellular, ubiquitous or tissue-specific, and mRNA stabilizing sequences such as polyadenilation sites. Examples of promoters used in the invention are: CMV IE (cytomegalovirus Immediate Early), SV40, TK (thymidino-kinase), or metallothioneine.

All therapeutic genes used in current vectors for gene therapy can also be used in the vectors according to the present invention: they therefore comprise genes or cDNA sequences or oligonucleotides replacing or correcting the defect of the corresponding mutated region in the genetic pathology, such as for instance muscular dystrophy, cystic fibrosis, hemophilia, omithine transcarbamylase (OTC) deficiency, X-linked immunodeficiency syndromes. Other therapeutic targets for gene transfer are infective or inflammatory pathologies, such as AIDS, amyotrophic lateral sclerosis (AML), rheumatoid arthritis, restenosis. Examples of therapeutic genes are sequences encoding cytokines, such as IL2, IL6, IL12, IL7 interleukines or TNF (tumor necrosis factor). Examples of therapeutic genes are also sequences encoding peptides, polypeptides or proteins with cytotoxic effect, for instance saporin, ricin or bacteria toxins. Therapeutic genes according to the present invention are also regulatory sequences transcribed within the cell though not necessarily translated into proteins, such as anti-sense, oligonucleotides blocking the translation of messenger RNAs which are over-expressed or dangerous for the cell or for a given period during the cell cycle.

The viral proteins or polypeptides which are used in order to prepare the chimeric vectors are chosen among: the HA protein (hemoagglutinin) of influenza virus, the VP22 protein of Herpes simplex virus, the penton-base adenoviral protein, the VP1 protein of FMDV (Foot and Mouth Disease Virus). It is possible to use also other proteins deriving from viruses such as: SV40, Cytomegalovirus, polyoma, FMDV, adeno-associated viruses (AAV), HepaDNAvirus, vaccinia virus, lentivirus or VSV (Vescicular Stomatitis Virus). Other examples of viral proteins are the envelopes of retroviruses such as HIV-1, HIV-2, RSV, MOMLV (Moloney Murine Leukemia Virus). The choice of the surface structural viral protein also depends on the desired target specificity, either species- or tissue-/cell-specific. In the case of a chimeric vector which should be targeted to CD4 lymphocytes, the bacteriophage might be engineered with the HIV-1 viral capsid. The interaction properties of microorganisms or viruses with the eukaryotic cell have also been demonstrated, in particular of the invasin protein of Yersinia pseudotubercolosis (Trau van Nhieu et al., 1993, EMBO J. 12:1887-1895) or of SA11 protein of Rotavirus (Hewish et al., 2000 J. Virol. 74:228-236).

Viral proteins and polypeptides also comprise mutants obtained by substitution, deletion, etc. of the aforesaid proteins, or their functionally analogous fragments. The criteria to be used in the choice of the viral protein modification or in the choice of the fragment can be depends upon the particular requirements of the chimeric phage, but they essentially aimed at: a) improving or maintaining the binding properties with respect to the integral protein, b) maintaining a stable structure when displayed, c) having a small size. The changes made to viral proteins and/or polypeptides may be also aimed at reducing the immunogenic nature of the viral protein or polypeptide.

According to a preferred embodiment the chimeric bacteriophage vector is chimerized with penton-base adenoviral protein, for instance belonging to Ad2 viral sub-type (SwissProt IDN: P03276) or to other adenoviral serotypes having equivalent binding properties, said protein being integral or deleted in N- and carboxy-terminal regions, or with its conservative mutants or serotype variants. According to this embodiment the phage vector acquires the binding specificity of the adenovirus, thus specifically binding to the eukaryotic cell and in particular to integrin receptors, and it also activates the internalization pathway typical of adenovirus which comprises the activation of phosphatidylinositol-3OH kinase (Li E et al. J Virol 1998; 72: 2055-2061), thus optimizing the vehiculation of the is therapeutic gene, of the reporter gene or of heterologous nucleic acids, through the cytoplasm to the nucleus.

According to another preferred embodiment, the chimerization of the bacteriophage is carried out with the polypeptide comprising the structurally stable central loop of the penton-base of Ad2. Said central loop preferably comprises amino acids 295-380 of penton-base (numbering system according to SwissProt IDN: P03276). Polypeptides having 6 to 10 consecutive amino acids of the penton-base protein may be also used for chimerization or, for instance, only particular amino acid strings containing binding patterns identified for integrin receptors, for instance strings containing RGD (Arg Gly Asp) and LVD (Leu Val Asp) patterns.

The authors of the present application have surprisingly found that the chimerized bacteriophage vectors according to these preferred embodiments not only acquire the binding specificity of the viral protein used for chimerization, thus binding specifically to the eukaryotic cell, but can also activate the internalization pathway which is typical of the virus as a whole, thus optimizing the vehiculation of the therapeutic gene, of the reporter gene or of heterologous nucleic acid, through the cytoplasm to the nucleus.

A further embodiment of the invention relates to a method for producing a bacteriophage vector chimerized with viral proteins or polypeptides derived from eukaryotic viruses expressed on the outer structure of the bacteriophage, essentially comprising the following steps:

i) introduction of a DNA sequence encoding for a viral protein or for a polypeptide of an eukaryotic virus into the genome of a bacteriophage or into a phagemid or into a plasmid;

ii) transformation of a bacterial host for the production of secreted or extruded phage particles in the culture medium;

iii) optionally infection with the whole phage;

iv) purification of chimeric phage particles.

The introduction of the sequence encoding for viral proteins or polypeptides is performed according to genetic engineering techniques known in the state of the art (Sambrook, J., E. F. Fritsch and T. Maniatis. 1989. Molecular cloning: a laboratory manual, 2^{nd} edition, Cold Spring Harbor Laboratory Press, N.Y.). It is performed either directly in the phage genome, or through the production of phagemids or plasmids which are used to transform bacterial hosts; in the latter case the production of phage particles requires the successive infection with the whole phage, be it a wild type phage or a helper phage or a phage containing modifications which are suitable for an “in-trans” expression strategy. An Example of such strategy is represented by the lambda bacteriophage containing an amber mutation in D or V protein, or any alternative strategy providing for the assembly within the host cell of proteins possibly also encoded by vectors (plasmid, bacteriophage and phagemid vectors) different from the bacteriophage. In the case of plasmids and phagemids the infection might optionally be replaced with transformation.

A further object of the invention is therefore represented by bacteriophage vectors and the chimeric phage particles obtained according to the process described. According to a first embodiment, said phagic particles acquire the capacity to bind eukaryotic cells specifically, due to the expression of proteins or polypeptides of eukaryotic viruses on their surface. In the process described the nucleotide sequences encoding the viral proteins or polypeptides are replaced or added to the genome of the bacteriophage, in the phagemid or in the plasmid by means of genetic engineering techniques known to people skilled in the art (Sambrook ibid.), so as to be displayed on the surface of the bacteriophage and to be functionally competent, for instance by maintaining the binding specificity of the native protein. Such nucleotide sequences are introduced into the sequences encoding the outer proteins of the bacteriophage, replacing them wholly or partially. In case of partial replacement the viral polypeptides or proteins are expressed as fusion proteins with phage proteins and then displayed on the phage surface.

Within the “naive” phage particle it is possible to identify outer protein structures or protein structures displayed on the surface, which are responsible for the bond to the natural target (consisting of bacteria). Said structures mainly consist of capsid proteins or of other bond-accessory proteins which are present on the outer surface of the bacteriophage.

The basic bacteriophage vectors used in the present invention comprise the genome of the bacteriophage, of phagemids or of plasmids, such as for instance λ-type bacteriophages, or M-13-type filamentous phages or phagemids and plasmids derived from the latter. When the bacteriophage is a lambda bacteriophage or comprises phagemids or plasmids derived from it, the chimerization takes place preferably on the outer D (capsid) or V (tail) phage proteins. The chimerization on the N-terminal portion of D protein is particularly preferred, said chimerization using an in-trans strategy enabling the production of phage particles containing up to 400 copies of the chimerized D protein and therefore allowing to obtain multivalent phage particles. The expression strategy makes use of a recombinant plasmid comprising a penton-base adenoviral protein or its fragments, or even more preferably the fragment corresponding to the central loop 286-393 of the adenoviral protein, which is used to transform bacteria infected with a lambda phage containing D protein under the control of an amber codon (Santi et al. 2000, J. Mol. Biol. 296:497-508). Among the advantages of the lambda phage vector with respect to other bacteriophage vectors we can quote: the capacity to accept large exogenous DNA sequences and the fact that its genome is double-stranded DNA.

According to a preferred embodiment of the invention, the chimerization of the bacteriophage with viral polypeptides or proteins is carried out by genetic engineering of the sequences encoding the proteins of the capsid pill, pVI or pVIII of M13, according to methods known to people skilled in the art (Hoogenbom H. R. et al. Nucleic Acid Res. 1991, 19:4133-4137). According to this preferred embodiment the nucleotide sequences encoding viral proteins or polypeptides are introduced in a partially deleted form of the sequence encoding pill protein of M13, contained into plasmids or phagemids known at the state of the art, such as those described in Savino et al. EMBO J. 1994, 13:1357-1367.

Surprisingly, when the chimeric phage particle is produced with the process according to the invention, the chimeric bacteriophage not only acquires the binding specificity of the viral protein used for chimerization, thus binding specifically to the eukaryotic cell, but it can also activate the internalization pathway which is typical of the virus as a whole, optimizing the vehiculation of the therapeutic gene, of the reporter gene or of heterologous nucleic acids, through the cytoplasm into the nucleus. According to a preferred embodiment, when the bacteriophage is chimerized with the integral or deleted penton-base protein of adenovirus 2 or their mutants, or serotypic variants in N- and carboxy-terminal regions, the chimeric bacteriophage according to the invention acquires the binding specificity of adenovirus, thus binding specifically to the eukaryotic cell, in particular to integrin receptors, and it activates the internalization way which is typical of adenovirus, comprising the activation of phosphaditylinositol-3OH kinase (Li E. et al. J. Virol. 1998; 72: 2055-2061), thus optimizing the vehiculation of the therapeutic gene, of the reporter gene or of heterologous nucleic acids, through the cytoplasm to the nucleus.

The properties of the specific binding of the adenoviral penton-base to the integrin receptor have been characterized in several studies and involve the regions containing RGD and LVD patterns, the latter being located in position 340-342 and 287-289 of penton-base, respectively, according to the numbering system used in SwissProt (IDN penton-base Adenovirus 2: P03276). Other sequences characterized in the penton-base adenoviral protein, such as endosomal escape sequences which confers resistance to internalized adenoviral sequences, active during the natural process of adenovirus infection, are also present in the chimeric phage according to the invention and optimize the whole transduction process.

Therefore, in a further embodiment the invention provides for a method of eukaryotic cells transduction, characterized in that it uses the chimeric vectors or the chimeric phage particles according to the invention.

When such process is based on the use of chimeric phage particles, the method for the transduction of eukaryotic cells provides, in addition to the steps described above:

i) insertion of a DNA sequence encoding a viral protein or a polypeptide of an eukaryotic virus into the genome of a bacteriophage or into a phagemid or into a plasmid;

ii) transformation of a bacterial host for the production of secreted phage particles;

iii) optionally infection with the whole phage;

iv) purification of chimeric phage particles, a further passage of

v) contacting of the purified phage particles with eukaryotic cells at a temperature between 25 and 39° C., preferably 37° C., still more preferably in presence of bivalent cations,

so that the phage particles bind to the eukaryotic cells and are internalized in a receptor-dependent way.

According to a further embodiment the bacteriophage vectors chimerized with penton-base adenoviral protein or with its central portion, bind eukaryotic cells expressing at least one type of integrin receptor. Therefore, a preferred embodiment of the invention provides for a method for the specific transduction of cells expressing at least one type of integrin receptor.

An additional feature of the invention provides for a method for identifying binding and internalizing mutants of penton-base adenoviral protein, substantially comprising the following steps:

i) random mutagenesis of the nucleotide sequence encoding penton-base adenoviral protein in a phagemid vector or in a phage genome or in a plasmid

ii) production of phage particles comprising the mutagenized sequences

iii) contacting of the phage particles with cells expressing integrins in selective conditions for the bonding or the internalization of the vector

iv) recovery of phagemid vectors after specific adhesion or internalization.

As an alternative, when the process aims at selecting mutants with improved binding capacity only, phage particles are contacted in the process according to the invention (Step iii) with purified integrins in such conditions as to enable the binding, for instance in presence of bivalent ions, such as Ca⁺⁺, and then selected by affinity.

The methods for random mutagenesis of DNA sequences are known in the state of the art (Sambrook et al.), as well as the production of phage libraries containing mutagenized heterologous sequences.

Selective conditions are those in which it is possible to detect an enrichment of a phage population with respect to another, for instance using for panning recombinant phages cells which can also be genetically engineered and which express only one type of integrin receptor.

Another embodiment of the invention is represented by compositions comprising a physiologically acceptable liquid and a bacteriophage vector for gene transfer into eukaryotes, said vector being chimerized with viral proteins or polypeptides chosen among: HA protein (hemoagglutinin) of influenza virus, VP22 protein of Herpes simplex virus, VP1 protein of FMDV virus or penton-base adenoviral protein or its fragments or conservative mutants or serotype variants. The penton-base adenoviral protein is the preferred choice. It is possible to use also other proteins deriving from viruses such as: SV40, Cytomegalovirus, polyoma, AAV, HepaDNAvirus, vaccinia virus, lentivirus or VSV (Vescicular Stomatitis Virus). Other examples of viral proteins are the envelopes of retroviruses such as HIV-1, HIV-2, RSV, MoMLV.

As an alternative, polypeptides are used consisting of fragments, possibly also mutagenized, obtained by conservative substitution or deletion of the aforesaid viral proteins and modified according to the following criteria: a) improve or keep binding properties with respect to the integral protein, b) keep a stable structure when displayed, c) have a small size. The changes made to viral proteins and/or polypeptides can also be carried out in order to reduce the immunogenic nature of the viral protein or polypeptide.

According to a preferred embodiment the composition comprises bacteriophage vectors chimerized with penton-base adenoviral protein or with its fragments, among which most preferred is the fragment comprising the central loop of penton-base. Said central loop preferably comprises amino acids 295-380 of penton-base (numbering system according to SwissProt). It is further possible to use polypeptides having 6 to 10 consecutive amino acids of penton-base protein, for instance peptides containing binding patterns identified for integrin receptors, for instance peptides comprising RGD (Arg Gly Asp) and LVD (Leu Val Asp) patterns.

Another feature of the invention comprises the use of the nucleotide sequence encoding penton-base adenoviral protein or its fragments or serotype variants, among which the most preferred is the sequence comprising amino acids 295-380 of penton-base adenoviral protein, to genetically engineer chimeric vectors for the transduction of eukaryotic cells.

The invention will now be disclosed through the following experimental examples which do not in any way limit its framework.

Experimental Part

### EXAMPLE 1

### Production of Chimeric Phages for Penton-Base Adenoviral Protein

The gene encoding the complete sequence of penton-base adenoviral protein (Pb) (SwissProt IDN P03276) and its central domain (ΔPb aa 286-393) were amplified by PCR from the DNA of Ad2 (Sigma, St. Louis Mo., USA) with the following pairs of primers:

(Pb 1-517):
5′-GATCGTCGACATGCAGCGCGCGGCGATGTATGAGG-3′/
5′-TGACGCGG CGCCCTAAAAAGTGCGGCTCGATAGGACGCGC-3′
and
(Pb 286-393):
5′-GATCGTCGACCTGTTGGATGTGGACGCCTACCAGGCA-3′/
5′-TGACGCGGCCGCCCTATAGGTTGTAACTGCGTTTCTTGCTGTC\-3′

and introduced into SalI-NotI site of pHenΔ phagemid (Savino et al. EMBO J. 1994, 13:1357-1367), in a position corresponding to the C-terminal portion of pill capsidic protein. Control phages containing no adenoviral sequence were also prepared. The expression box CMV-GFP-polyΔ for the expression of the eukaryotic protein Green Fluorescent Protein was obtained by digestion of pITRUF5-N plasmid (PNAS 1999 Recchia A. et al., 96:2615-2620) with EcoRI and Sail enzymes, made blunt-ended and then sub-cloned in the recombinant phagemids Pb(1-571)-pHenΔ and Pb(286-393)-pHenΔ.

The sequence and extension of the deletion of penton-base adenoviral protein in the chimeric phage were designed considering several parameters for the production of ΔPb chimeric phage: i) minimization of insert size to limit possible interference in the assembly of DNA and of the phage capsid, ii) inclusion within the fragment of Pb-binding pattern RGD for integrins, iii) stability of deleted insert, evaluated both on the basis of literature data (Stewart PL et al. EMBO J 1997; 16: 1189-1198) and by predicting the secondary structure with Predict Protein (EMBL server, Heidelberg). As a whole, the analyses referring to structure and alignment with adenoviral sequences of several serotypes through the Pile-up program (Genetic Computer Group package, used with default parameters) showed that the fragment of Pb 286-393 (ΔPb) has a stable structure containing RGD pattern surrounded by alpha-helixes.

The phages were prepared by transformation of E. Coli XI1-blue (Stratagene, La Jolla, Calif., U.S.), super-infected with helper phage M13 KO7 (Amersham-Pharmacia Biotech, Uppsala, Sweden) and the secreted phage form was PEG-precipitated from the supernatant of E. coli, then further purified by ultra-centrifugation on CsCI gradient.

The expression of pb and Δpb adenoviral protein on the phage capsid was verified by western-blotting of the chimeric phage particles purified from the culture medium of infected bacteria. After SDS-PAGE and blotting onto nitrocellulose the filter was saturated with TBS/5% powder milk/0.05% Tween 20 (TBSMT) and incubated with an anti-Pb rabbit polyclonal antibody diluted 1:1000 in TBSMT, and the bound antibody was detected with an anti-rabbit antibody conjugated with HRP (Amersham-Pharmacia Biotech) after development with ECL system (ECL+system kit antibody, Amersham-Pharmacia Biotech). The results of western-blot analysis showed that the chimeric proteins are expressed on the phage with the expected molecular weight and that the chimeric proteins were also recognized by specific anti-penton-base antibodies. The chimeric proteins on the phage capsid were further quantified using as controls a sample consisting of 1.2×10⁹ (corresponding 7.2×10¹⁰ molecules of Pb) and a control consisting of 1.2×10¹⁰ phage particles (corresponding to 4.8×10¹² molecules of pIII) and measuring the corresponding signal intensity with the program Phoretix1. The relative quantification showed that the chimeric protein ΔPb-pill is expressed in 1/20 particles of the phage stock, whereas the chimeric protein corresponding to the complete form of Penton-base Pb-pIII protein was expressed in 1/90 of the phage stock.

### EXAMPLE 2

### In-Vitro Bond of the Chimeric Phages to the Integrins

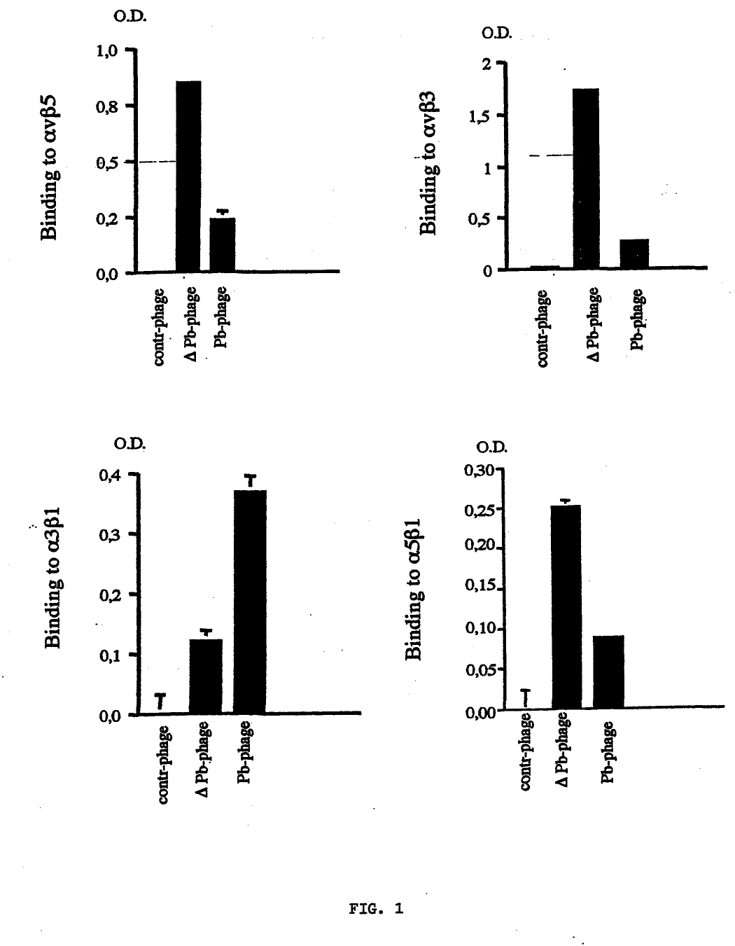

In order to verify whether the phage structure allows the functional expression of the integral and deleted penton-base protein and whether the latter keeps the binding properties to integrins which are typical of the integral protein, the binding of the chimeric phages to purified integrins αvβ3, αvβ5, α5β1 and α3β1 (Chemicon, Temecula Calif.) was measured in vitro. 96-well plates were coated with the purified proteins for 16 hours at 4° C., then washed and blocked in TBSMT added with Ca⁺⁺ (TBSMT+). The chimeric phages were added to the wells and incubated for 2 hours. In competition tests, said coated wells were pre-incubated 1 hour before the addition of the phages with GRGDSP or GRGESP peptides (Sigma, St. Louis, Mo.). The phages bound to the plate were detected with an anti-M13 monoclonal antibody for pVIII protein (Amersham-Pharmacia Biotech), diluted 1:500 in TBSMT+. After incubation with a secondary HRP-conjugated anti-mouse antibody (Amersham-Pharmacia Biotech), diluted 1:1000 in TBSMT+, the HRP signal was developed with a TMB substrate (Sigma, St. Louis, Mo.) and the optical density read at 450 nm with Microplate Reader (Biorad, Hercules, Calif.). FIG. 1 shows the results of the ELISA assay: both chimeric (ΔPb and Pb) phages can bind to all tested integrins and in particular to αvβ3, αvβ5, α3β1 and α5β1 integrins, though at a different levels, in particular the ratio of the signal of Pb phage to the signal of ΔPb phage varies from ¼ to ⅕ when the binding was measured on αvβ3, αvβ5 and α5β1 integrins, whereas said ratio is inverted (5:1) when the binding was measured on α3β1 integrin. As expected, no signal was observed after incubation with control phages.

Table 1 shows the results of the competition tests with GRGDSP and GRGESP peptides.

TABLE 1
Binding specificity of chimeric phages to integrin receptors

| | | Binding | Binding | Binding |
|---|---|---|---|---|
| Competitor | | to αvβ3 | to αvβ5 | to α3β1 |
| peptide | Phage | (% control) | (% control) | (% control) |
| GRGDSP | ΔPb | 14 | 70 | 37 |
| | Pb | 25 | 58 | 21 |
| GRGESP | ΔPb | 93 | 94 | 95 |
| | Pb | 62 | 105 | 99 |

The test was briefly carried out as follows: 10¹² phage particles were incubated with purified integrins (0.2 μg/ml) in presence or absence of competitor peptides (4.86 μM).

The results were expressed as percentages of the binding obtained in absence of the competitor peptide and show that, the peptide GRGDSP, but not the control peptide GRGESP, reduced significantly the binding of both phages to the integrins on the plate. The data demonstrate the specificity of binding between both chimeric phages and integrins. The data also point out the strict dependence on the RGD pattern of the interaction between ΔPb phage and αvβ3 integrin.

### EXAMPLE 3

### In-Vivo Internalization of Chimeric Phages

The capacity of the phage to bind and to be internalized in vivo was verified by electron microscopy (a) and immunofluorescence (b) on HeLa was cells. The quantification of binding and internalization levels of the chimeric phages was carried out by micropanning on HeLa, CS-1 and CS-1/β3 cells (c).

Cell Cultures

Hela cells (ATCC no. CCL-2) are grown in DMEM/10% fetal calf serum (FCS). CS-1 and CS-1/β3 are grown in RPMI/10°/, for the second group of cells with the addition of G418 (Genetycin, Sigma, St. Louis, Mo.) 500 μg/ml.

a) Electronic Microscopy

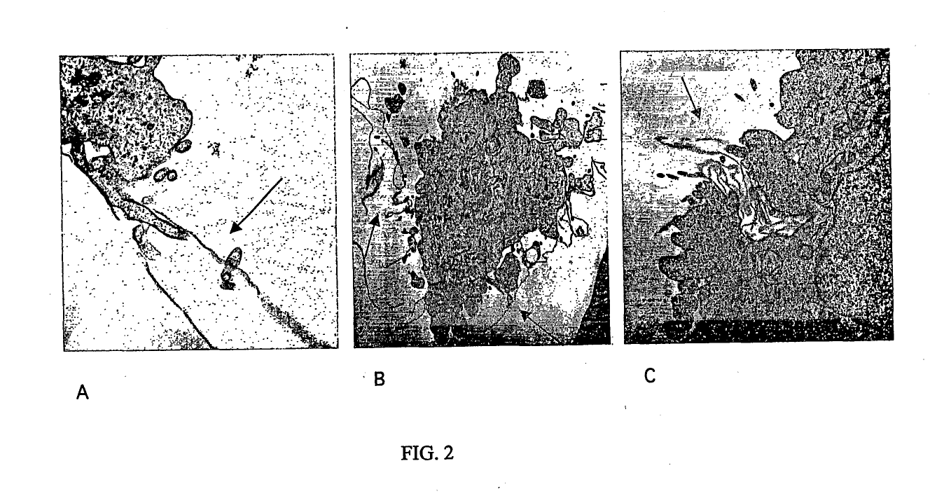

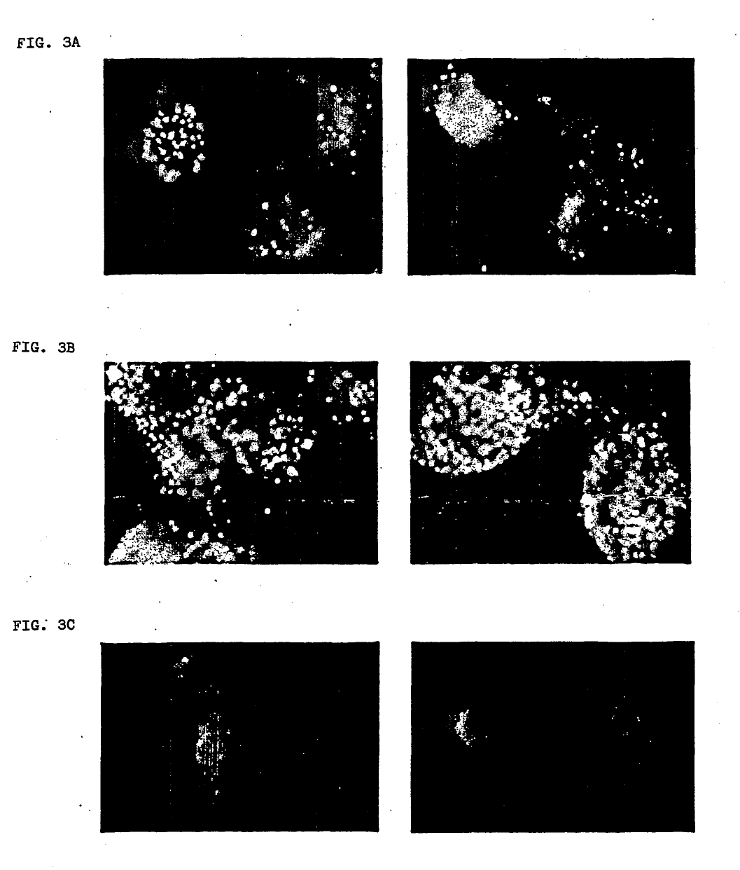

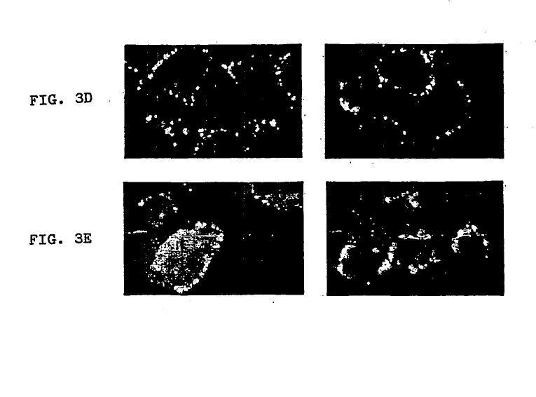

10⁵ HeLa cells were plated 48 hours before and incubated with 3×10¹² ΔPb phages and 9×10¹² Pb phages, respectively, and incubated for 1 hour at 4° C. After being washed the cells were fixed for 10′ in 2% glutaraldehyde in 0.15 M Hepes buffer at pH 7.3 and fixed for electronic microscopy for 1 hour at 24° C. in a solution of 0.1 M cacodylate buffer, pH 7.3, containing 1% osmium tetroxide and 1.5% potassium ferrocyanide. The cells underwent dehydration and were then immersed in Epon 812 resin. Ultra-thin sections were dyed with uranyl acetate and lead citrate and observed with an electronic transmission microscope (CM100; Philips, Eindhoven, The Netherlands). FIG. 2 shows the pictures obtained with Pb phage (FIG. 3A) and with DPb phage (FIGS. 3B and 3C), which are visible after the washing steps as filamentous structures present on the surface and in the invaginations of the cell membrane and which are absent in the control.

b) Immunofluorescence

2.5×10⁵ HeLa cells were plated in DMEM/10% FCS 48 hours before the incubation with the chimeric phages at 4° C. in PBS+/5% FCS. When indicated 100 μM chlorokine (Sigma) was added and the incubation was continued for 1 hour at 37° C. After few washings with cold PBS+/5% FCS the cells were fixed in PBS+/3.7% formaldehyde and permeated, when necessary, with PBS+/0.1% Triton X-100. After some washings in PBS/0.01% Tween 20/5% powder milk (blocking buffer: BB) the cells were incubated with an anti-M13 monoclonal mouse antibody (Amersham-Pharmacia Biotech), diluted 1:50 in BB, and after some more washings further incubated with an antibody conjugated with anti-mouse FITC (Dako, Denmark), diluted 1:50, and analyzed with a fluorescence microscope with a 40× objective. The pictures were taken with a digital camera (CCD) using suitable filters, and visualized with Adobe-Photoshop software.

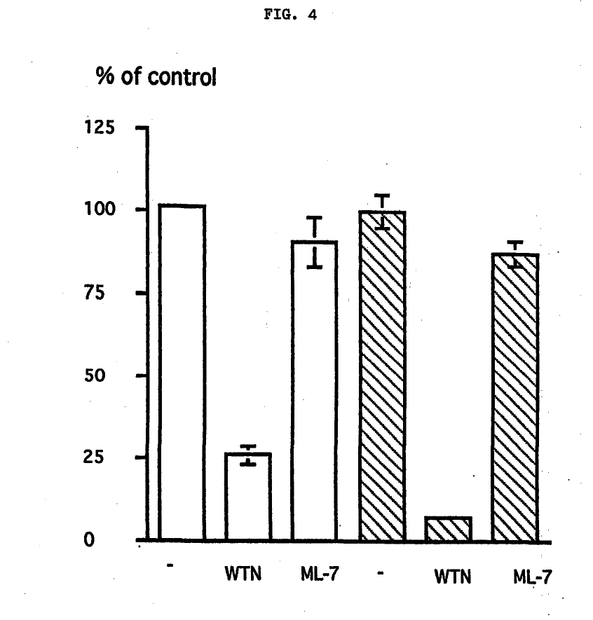

FIG. 3 shows the results obtained with Pb phage (FIG. 3A) and with ΔPb phage (FIG. 3B): the fluorescence is visible on the cytoplasm, thus showing the internalization of the phage. On the other hand, FIGS. 3D and 3E show the results obtained by incubating ΔPb phage at 4° C. At this temperature the receptor-mediated endocytosis is blocked, the latter being an energy-consumption mechanism, and therefore the fluorescence remains outside the cell (FIGS. 4D and E, respectively) independently or not on the cell permeation chemically induced before adding the antibody. At all tested temperatures the fluorescence intensity of the tests carried out with the control phage is not significant, thus showing the absence of internalization.

c) In-Vivo Phage Micropanning

The micropanning on HeLa and CS-1 or CS-1/β3 cells was carried out in vivo at 4° C. and at 37° C. in order to quantify the bond and the internalization of chimeric phages, respectively. When evaluating micropanning only for internalization, the washings after the incubation of phage particles on the cells were carried out in 6M Urea/1N HCl, so as to eliminate all non-internalized phages. In short, 7.5×10⁴ cells were plated on trays containing 24 wells 48 hours before the experiment. Chimeric phages Pb and ΔPb were then incubated at 4° C. in PBS+/5% FCS. The phages still bound to the cells after the washings, were eluted in 6M Urea/1N HCl/pH 2.2, 4° C. for 10′. The eluates were titrated by ELISA on a plate coated with an anti-M13 antibody detected with anti-pVIII-HRP antibody, using as standard a phage stock with known titer.

Phage internalization was evaluated on cells which have been pre-incubated for 30′ at 37° C. with chlorokine 100 nM (Sigma) alone or together with an inhibitor of PI3K (phosphatidylinositol-3OH kinase), 1 μM Wortammannin (Sigma) or with an inhibitor of myosin light chain kinase ML-7 2 μM hydrochloride (Calbiochem, La Jolla, Calif.).

The phages, diluted in PBS+/5% FCS and 100 nM chlorokine, with or without the aforesaid kinase inhibitors, were added to the cells and incubated for 1 hour at 4° C., then for 2 hours at 37° C., and successively eluted with 6M Urea/1N HCl/pH 2.2. The cells were lysed in 10 mM TrisHCl/2 mM EDTA/2% DOCNa/pH 8.0 and the phage titer was measured in the cell lysate by infection of bacterial cells. The results of these experiments are shown in FIG. 4. In particular, it is shown how the activation induced by the bacteriophages according to the invention, chimerized both with the integral penton-base adenoviral protein and with its deleted form, ΔPb, activates phosphatidylinositol-3OH after binding to the cell, and their internalization is inhibited by Wortammannin but not by MAP-kinase or by myosin light chain kinase which is inhibited by ML-7. This kinases activation pattern, which is different from the one of natural integrin ligands activating Erk1/Erk2 kinases, corresponds to the typical activation pattern used by adenovirus during the natural infection process through αv-integrin way. Therefore, it indicates that the chimeric phages according to the invention acquire the ability of being internalized in a similar way to integral adenovirus.

In order to give a correct evaluation of the micropanning results, in particular as far as the binding efficiency of the phages to the cells is concerned, the cell systems used (HeLa, CS-1 and CS-1/β3) were previously analyzed by FACS so as to evaluate the expression levels of integrin receptors. Such analyses showed that CS-1 cells are not positive for any kind of integrin, CS-1/β3 are, as expected, highly positive to an anti-αvβ3 antibody (72%), whereas HeLa cells in the conditions applied are negative for integrins belonging to αvβ3 and αvβ5 subtypes, though strongly positive (67.3%) for integrins belonging to β1 subtype. The results of the micropanning experiments are shown in table 2.

TABLE 2
Differences of phage enrichment measured in vivo

| | |
|---|---|
| Bond | Internalization |

| | Pb | ΔPb | Pb | ΔPb |
|---|---|---|---|---|
| HeLa | 8 ± 3 | 15 ± 6 | 9 ± 3 | 40 ± 16 |
| CS-1 | 1 | 1 | 1 | 1 |
| CS-1/β3 | 4 ± 1 | 77 ± 19 | 2 ± 1 | 12 ± 3 |

In short, the experiment was carried out as follows: 7.5×10⁴ cells/well are plated and incubated with 10¹² chimeric phage particles. The results are expressed as enrichment factors with respect to control phages. Each experiment is carried out on two series and repeated twice: the results are expressed as inter- and intra-assay average values. Standard deviation is indicated.

The results shown in table 2 show that chimeric phages for the integral or deleted penton-base protein can be selected in vivo in selective conditions both for the binding and the for internalization into the eukaryotic cell. Furthermore, the data obtained in this experiment are in agreement with the observations made in vitro and with preliminary experiments involving integrin expression, and confirm them. They indeed prove that the lowest ratio of ΔPb/Pb-phage is observed in HeLa cells, further suggesting the possibility of preferential vehiculation towards integrins, or towards cells expressing integrins, belonging to αvβ3, αvβ5 and α5β1 subtypes with the chimerized phage ΔPb (adenoviral protein with deleted N- and C-terminal portions), and on integrins belonging to α3β1 subtype (or on cells expressing said integrins), with the Pb phage chimerized with the integral penton-base protein.

From the data obtained by the western-blot described in example 1 and from the internalization data shown in table 2, it can be inferred that the deletion of penton-base for the production of ΔPb-phage increases the display efficiency on the phage particle, maintaining, or even increasing as shown in the internalization experiments carried out on some cell types, the functional properties of the integral penton-base.

### EXAMPLE 4

### Evaluation of Transduction Efficiency of Eukaryotic Cells (HeLa, CS-1 and CS-1/β3) with Chimeric Phages by Expression of GFP Reporter Gene

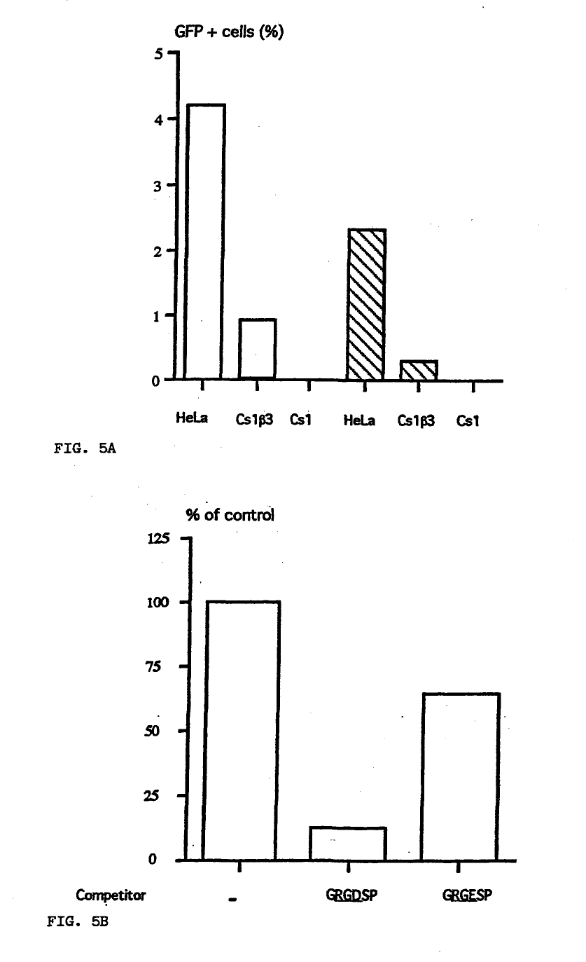

The expression of GFP reporter gene (Green Fluorescent Protein) was carried out by FACS analysis as follows: 1×10⁵ cells (HeLa, Cs-1/β3 or Cs-1) were plated in 6-portion wells. After 24 hours the cells were incubated for 1 hour at 4° C. and for 3 hours at 37° C. with 2×10¹³ chimeric particles (Pb-GFP and ΔPb-GFP phage) containing the gene for GFP. After the washings and 72 hours of incubation in fresh medium the expression levels of GFP protein were measured by FACS. The specificity of the measured signal was evaluated also in presence of GRGDSP or GRGESP peptides, pre-incubating the cells for 1 hour, with peptide concentration of 4.86 μM, corresponding to a molar excess of about 2000 times and maintaining said concentrations in the following incubations. 10⁴ cells were measured for each sample. As can be inferred from the data, chimeric phages caused an effective expression both in HeLa cells and in Cs-1/β3 cells; however, the best transduction effectiveness was obtained in HeLa cells transduced with ΔPb-GFP cells (>4°/) FIG. 5a. In CS-1 cells which lacks integrin receptors, as expected, no GFP expression is observed. FIG. 5b shows the data concerning the specificity of the transduction mediated by the integrin receptor: GRGDSP peptide inhibits up to 90% the transduction of HeLa cells by ΔPb-GFP phage, which is not observed with control peptide GRGESP.

As a whole, the data therefore show that both Pb and ΔPb phage can be used as vectors in gene therapy of eukaryotic cells, since they bind specifically and in a way competible by specific peptides, to integrins expressed in vivo on the cells. They can also vehiculate them through the cytoplasm into the nucleus, enabling the expression of exogenous DNA sequences, as proved by the effective expression of the reporter gene GFP.

Considering the ubiquitarian expression of integrin receptors, said vectors can therefore be used for a large group of applications of gene therapy.

### EXAMPLE 5

### Production of Chimeric Lambda Phage

The gene encoding the complete sequence of penton-base adenoviral protein (Pb).(SwissProt ION P03276) and its central domain (ΔPb: aa 286-393) were amplified by PCR from the DNA of Adenovirus serotype 2 (Sigma, St. Louis Mo., USA) with the following pairs of primers:

(Pb 1-571)
5′-GATGCCATGGCAATGCAGCGCGCGGCGATGTATGA-3′/
5′-GATGCCATGGAAAAAGTGCGGCTCGATAGGACG-3′ and
(Pb 286-393):
5′-GATGCCATGGCACTGGATGTGGACGCCTACC-3′/
5′-GATGCCATGGTTAGGTTGTAACTGCGTTTCTT-3′,

and introduced into the NcoI site of pNS3785 plasmid (Sternberg REF 1995 PNAS 92, p. 1609-1613), in a position corresponding to the N-terminal portion of D capsid protein, resulting in pNS3785Pb and pNS3785ΔPb plasmids, respectively. The lambda phage containing the reporter gene GFP was produced starting from p171 IoxP- lambda phage (Santi, J. Mol. Biol., 296, page 497-508, 2000). The lambda genome p171 IoxP- was digested with SacI, thus resulting in the elimination of the second site IoxP and of p171 plasmid.

The restriction sites SfiI, AvrII, SwaI, PacI were then introduced into SacI site with the following oligonucleotide:

5′-GGCCCATATGGCCTAGGATTTAAATTMTTAAAGCT-3′.

The genome thus modified was digested with SwaI so as to introduce the CMV GFP polyA box (derived from plasmid pITRUF5-N, PNAS 1999 Recchia A. et al., 96:2615-2620), and upstream to this the sequence for nuclear localization (5191-311) deriving from SV40.

A trans-expression strategy was used to prepare chimeric phages. As a matter of fact, the chimeric plasmid for the sequence encoding adenoviral penton-base was used to transform bacteria infected with a lambda phage carrying an amber mutation in D protein. This strategy enabled the assembly of the chimeric protein on the mature phage and the production of λ bacteriophages with the higher number of copies (around 400) of chimeric protein on their surface.

In further detail, chimeric phages were prepared by transformation of BB4 bacterial stock (Stratagene) with pNS3785 ΔPb/pNS3785Pb plasmid and infection with SV40 CMV GFP polyA lambda phage was later performed. Bacterial lysates were treated with NaCl and precipitated with PEG, and then bacteriophage particles were purified by ultra-centrifugation on CsCI gradient.

Control phages containing no adenoviral sequences were also prepared.

### EXAMPLE

### Binding of Chimeric λ Bacteriophages to Integrins

The binding of chimeric lambda phages to integrin receptors and the incorporation of the deleted recombinant protein penton-base (ΔPb-D) on the capsid of lambdaΔPb phages were evaluated by ELISA assay. 96-well plates were coated with purified integrin ανβ3 (0.1 μg/well) (Chemicon, Temecula Calif.) for 16 hours at 4° C., and then washed and blocked in TBSMT added with Ca⁺⁺ (TBSMT+). Chimeric lambda phages were added to the wells (10⁷/10⁸ pfu/well) and left under incubation for 2 hours. After 6 washings with TBST+ the phages which were still bound to the integrins were detected with an anti-lambda polyclonal antibody (diluted 1:2000 in TBSMT+).

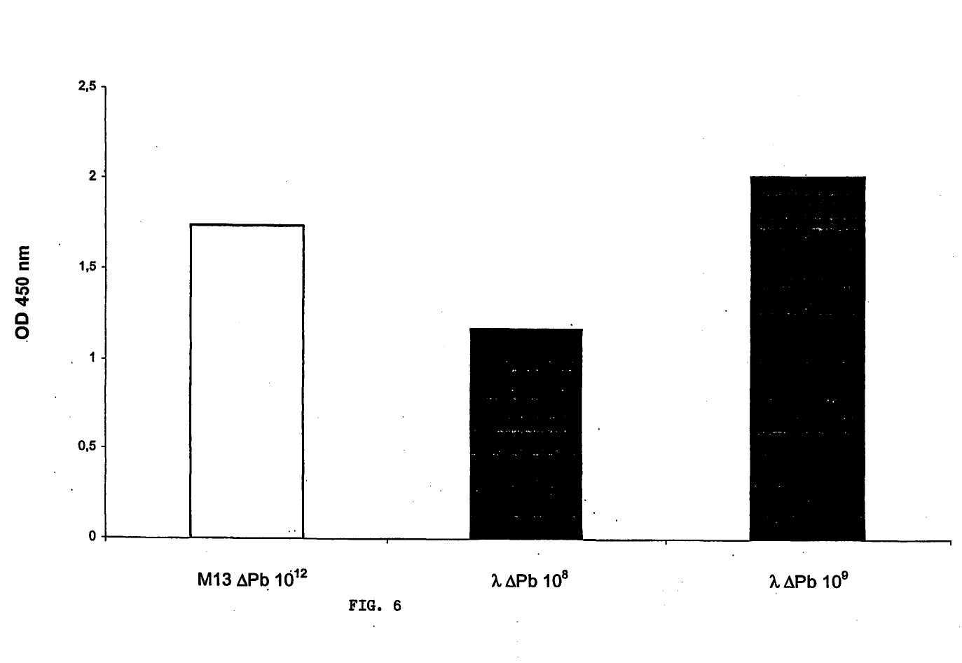

After incubation with a conjugated anti-rabbit HRB secondary antibody (Amersham-Pharmacia Biotech), diluted 1:1000 in TBSMT+, the HRP signal was observed with TMB substrate (Sigma, St. Louis, Mo.) and the optical density is read at 450 nm with ELISA Microplate Reader (Biorad, Hercules, Calif.). In the ligand-binding experiment whose results are shown in FIG. 6, the binding efficiency to ανβ3 integrin of filamentous chimeric phage particles (M13) and of lambda, in an amount of 10¹² and of 10⁸-(10⁹), respectively, was compared. results shown in FIG. 6, demonstrate that similar extent of binding to the integrin, directly proportional to the optical density measured with ELISA assay, is obtained, within the same assay with 10¹² particles of M13 ΔPb phage and with 10⁹ of lambda. This significant increase (at least three times as much) in the affinity and/or of the avidity of the phage particle can be explained through the polyvalence of the lambda phage particle with respect to the particle of the filamentous phage M13.

1. Bacteriophage vectors for gene transduction of eukaryotic cells, characterized in that they are chimerized with polypeptides or proteins of eukaryotic viruses.

2. Chimeric bacteriophage vectors according to claim 1, further comprising a therapeutic gene under the control of a transcription promoter.

3. Chimeric bacteriophage vectors according to claims 1-2, wherein said viral polypeptides or proteins are structural proteins of eukaryotic viruses comprised in the group consisting of: influenza virus, herpes viruses, retroviruses, polioma virus, SV40, adenoviruses and adeno-associated viruses, vaccinia virus, lentivirus.

4. Chimeric bacteriophage vectors according to claims 1-3, wherein said viral polypeptides or proteins are comprised in the group consisting of: VP22 of herpes virus HSV, hemoagglutinin of influenza virus, adenoviral penton-base.

5. Chimeric bacteriophage vectors according to claim 4, wherein said viral polypeptide or protein comprises amino acids 1-571 of adenoviral penton-base is (SwissProt IDN:P 03276) or its serotype variants or its conservative mutants.

6. Chimeric bacteriophage vectors according to claim 5, wherein said viral polypeptide or protein comprises at least one polypeptide fragment of the adenoviral penton-base or its serotype variants or its conservative mutants.

7. Chimeric bacteriophage vectors according to claim 6, wherein said viral polypeptide or protein comprises amino acids 295-380 of the adenoviral penton-base or its serotype variants or its conservative mutants.

8. Chimeric bacteriophage vectors according to claim 6, wherein said polypeptide fragments comprise 6 to 10 consecutive amino acids of adenoviral penton-base protein.

9. Chimeric bacteriophage vectors according to claims 1-8, wherin the bacteriophage vector is a filamentous phage M13 or its phagemids.

10. Chimeric bacteriophage vectors according to claims 1-8, wherin the bacteriophage vector is a lambda bacteriophage or its phagemids or plasmids.

11. Process for the preparation of chimeric phage particles comprising the following steps:
i) introduction of a DNA sequence encoding a viral protein or a polypeptide of an eukaryotic virus into the genome of a bacteriophage or into a phagemid or into a plasmid;
ii) transformation of a bacterial host for the production of chimeric phage particles;
iii) optionally infection with the whole phage;
iv) purification of chimeric phage particles.

12. Process according to claim 11, wherein the genome of the bacteriophage or the phagemid or the plasmid further comprises a therapeutic gene under the control of a transcription promoter.

13. Process according to claims 11 and 12, characterized in that the introduction into the genome of the bacteriophage or into the phagemid or into the plasmid as in step i) of the process occurs at the level of the DNA sequence encoding for a structural protein of the phage particle.

14. Process according to claim 13, wherein said structural phage protein is chosen among: pill or pVIII capsidic protein of M13, D capsid protein of lambda phage or V protein of the tail of lambda phage.

15. Process according to claim 14, wherein said structural phage protein is the M13 capsidic protein pill or the capsidic D protein of lambda phage.

16. Process according to claims 11-15, wherein the DNA sequence encoding viral proteins or polypeptides comprises the nucleotide sequence encoding amino acids 1-571 of adenoviral penton-base or its conservative mutants or its serotype variants.

17. Process according to claims 11-15, wherein the DNA sequence encoding for viral proteins or polypeptides comprises the nucleotide sequence for encoding amino acids 295-380 of the adenoviral penton-base or its conservative mutants or its serotype variants.

18. Process according to claims 11-15, where the DNA sequence encoding viral proteins or polypeptides comprises a nucleotide sequence encoding at least 6 consecutive amino acids of the adenoviral penton-base or its conservative mutants or its serotype variants.

19. Process according to claims 11-18, characterized in that the genome of the bacteriophage, as in step i) of the process is the genome of the filamentous phage M13 mp or one of its phagemids.

20. Process according to claims 11-18, characterized in that the genome of the bacteriophage as in step i) of the method is the genome of the lambda phage or one of its phagemids or plasmids.

21. Chimeric phage particles, obtainable by the process according to claims 11-20.

22. Process for the transduction of eukaryotic cells, characterized in that the chimeric bacteriophage vectors according to claims 1-10 are used.

23. Process for the transduction of eukaryotic cells, characterized in that the chimeric phage particles according to claim 21 are used.

24. Process for the transduction of eukaryotic cells according to claim 23, characterized in that it comprises the following step:
v) contacting of the chimeric phage particles according to claim 20 with eukaryotic cells at a temperature between −25 and 39° C., so that the vector is internalized.

25. Process for the transduction of eukaryotic cells expressing at least one type of integrin, characterized in that the chimeric vectors according to claims 5-10 or the phage particles obtained with the process according to claims 16-20 are used.

26. Procss according to claim 25, where said integrin is chosen among: ανβ3, αvβ5, α5β1 and α3β1 integrins.

27. Process for identifying binding and internalization mutants of a penton-base adenoviral protein, essentially comprising the following steps:
i) random mutagenesis of the nucleotide sequence encoding penton-base adenoviral protein in a phagemid vector or in a phage genome or in a plasmid
ii) production of chimeric phage particles comprising the mutagenized sequences
iii) contacting the chimeric phage particles with cells expressing integrins in selective conditions
iv) recovery of chimeric phagemid vectors selected after specific adhesion or internalization.

28. A composition comprising a physiologically acceptable liquid and a bacteriophage vector according to claims 1-10 or a phage particle according to claim 21.

29. Use of the nucleotide sequence encoding penton-base adenoviral protein (SwissProt IDN:P03276), its fragments, serotype variants or conservative mutations, for the preparation of chimeric vectors for the transduction of eukaryotic cells.

30. Use according to claim 29, where said fragment comprises the sequence encoding amino acids 295-380 of penton-base adenoviral protein (SwissProt IDN:P03276), or its serotype variants.
